(19) United States
(12) Patent Application Publication
(10) Pub. No.: US 2021/0113479 A1
(43) Pub. Date: Apr. 22, 2021

(54) DOSAGE FORMS FOR DELIVERY OF MEDICINES TO THE LOWER GASTROINTESTINAL TRACT

(71) Applicant: M.W. Encap Limited, Livingston West Lothian (GB)

(72) Inventors: Wei Tian, Cleghorn (GB); Graeme William Andrew Hamilton Johnston, Blantyre (GB)

(21) Appl. No.: 17/052,036

(22) PCT Filed: May 3, 2019
(86) PCT No.: PCT/EP2019/061329

§ 371 Date: Oct. 30, 2020

(30) Foreign Application Priority Data
May 3, 2018 (EP) 18170574.0

(51) Int. Cl. A61K 9/48; A61K 31/522

(52) U.S. Cl. CPC A61K 9/4891; A61K 9/4825; A61K 9/4866; A61K 31/522

(57) ABSTRACT

The instant disclosure provides a capsule suitable for delivery of medicines to the lower gastrointestinal tract.

### FIELD OF THE INVENTION

The present application relates to capsules capable of providing delivery of active ingredients to the lower gastrointestinal tract, such as the distal small intestine, the large intestine and colon.

### BACKGROUND

Delivery of active ingredients in dosage forms suitable for oral administration to the lower gastrointestinal tract, such as the distal small intestine, large intestine or colon, has been attempted using a variety of approaches. One approach is to design a dosage form in which the dosage form releases the active ingredient at a particular time. This approach attempts to take advantage of the transit time required for the dosage form to move through the gastrointestinal tract to the desired area of release. Another approach is to design dosage forms that release the active ingredient at a particular pH. This approach attempts to take advantage of the increase in pH from the stomach to the distal small intestine. Yet another approach is to design dosage forms that can be digested in the large intestine by the flora present in that area of the gastrointestinal tract. However, all of these approaches are subject to variability of release of the active ingredient, with some patients (depending on the approach) experiencing early release (for example due to slow transit time) or no release at all due to fast transit time or a relatively low pH environment.

What is therefore desired is a dosage form that releases a relatively small amount of the active ingredient in the stomach and small intestine, and primarily releases the active ingredient in the lower gastrointestinal tract (such as the large intestine, colon and/or rectum), and is capable of having the release rate of the active ingredients easily adjusted so that the active ingredients may be administered to the desired portion of the lower gastrointestinal tract.

### SUMMARY

In one aspect a dosage form for colonic delivery comprises a capsule containing a fill composition and a coating over the capsule. The coating comprises a low pH polymer and a high pH polymer. The low pH polymer dissolves in a phosphate buffer solution at a pH of greater than 4.5 and less than 7. The high pH polymer dissolves in a phosphate buffer solution at a pH of greater than 6.8. The weight ratio of the low pH polymer to the high pH polymer is from 1:20 to 20:1, preferably from 1:15 to 15:1, more preferably from 1:10 to 10:1, or from 1:6 to 6:1, or from 1:5 to 5:1, most preferred from 1:4 to 4:1 (wt low pH polymer:wt high pH polymer). In another embodiment, the weight ratio of the low pH polymer to high pH polymer in the coating may be from 1:3 to 3:1, and may be from 1:2 to 2:1 (wt low pH polymer:wt high pH polymer).

In one aspect, the dosage form provides a lag time for release of the active ingredient of less than 20 wt % of the active ingredient, more preferably less than 10 wt %, within the first 30 minutes after administration to a phosphate buffer solution when tested at pH 6.8 and 37° C.

In another aspect, the low pH polymer dissolves at a pH of greater than pH 5, preferably greater than pH 5.5, and is dissolved at a pH of less than pH 7, preferably is dissolved at a pH of less than pH 6.8.

In another aspect the low pH polymer is selected from polymethacrylates, polyvinyl acetate phthalate, hydroxypropylmethyl cellulose acetate succinate (HPMCAS), hydroxypropylmethyl cellulose phthalate (HPMCP), and carboxymethyl ethyl cellulose.

In another aspect, the low pH polymer is selected from copolymers of methyl methacrylate and methacrylic acid, and copolymers of ethyl acrylate and methacrylic acid.

In another aspect the high pH polymer dissolves at a pH of greater than 7, or may dissolve at a pH of greater than 7.2.

In another aspect the high pH polymer is selected from polymethacrylates, HPMCAS and shellac.

In another aspect, the high pH polymer is selected from copolymers of methyl methacrylate and methacrylic acid, copolymers of ethyl acrylate and methacrylic acid, and poly(methacrylic acid, methyl acrylate, methyl methacrylate).

In another aspect the weight ratio of the low pH polymer to the high pH polymer is from 1:10 to 10:1, may be from 1:5 to 5:1, and preferably from 1:4 to 4:1 (wt low pH polymer:wt high pH polymer).

In one aspect the coating further comprises a plasticizer.

In one aspect the coating further comprises a glidant.

In one aspect, the coating comprises the low pH polymer in an amount of from 15 wt % to 50 wt %, the high pH polymer in an amount of from 15 wt % to 50 wt %, a plasticizer in an amount of from 5 wt % to 15 wt %, and a glidant in an amount of from 20 wt % to 40 wt % (wt % on a dry coating basis). In a preferred embodiment, the coating comprises the low pH polymer in an amount of from 40 wt % to 50 wt %, the high pH polymer in an amount of from 10 wt % to 20 wt % (wt % on a dry coating basis). In another preferred embodiment, the coating comprises the low pH polymer in an amount of from 10 wt % to 20 wt %, the high pH polymer in an amount of from 40 wt % to 50 wt % (wt % on a dry coating basis). In yet another embodiment, the coating comprises the low pH polymer and the high pH polymer each in an amount of from 25 wt % to 35 wt % (wt % on a dry coating basis). Any one of the polymers listed herein can be used in said coating compositions. In a particularly preferred embodiment the low pH polymer is a polymethacrylate (such as Eudragit L30 D-55 (e.g. approximately 30% dry solid)) and the high pH polymer is a polymethacrylate (such as Eudragit FS 30D (e.g. approximately 30% dry solid)).

In one aspect the capsule is banded or sealed.

In one aspect the dosage form further comprises a subcoating over the capsule.

In one aspect, the capsule comprises gelatin, HPMC, pullulan, or starch,

In one aspect, the fill composition is a sustained release composition.

In one aspect the fill composition comprises a gelling or swelling agent suspended in a water soluble or water dispersible non-aqueous matrix. In a preferred embodiment, said gelling agent is present in an amount of from 80 wt % to 95 wt % and/or the swelling agent is present in an amount of from 5 wt % to 20 wt %.

In one aspect, the fill composition comprises a highly water soluble agent suspended in a water insoluble waxy matrix.

In one aspect, said dosage form does not comprise an oligonucleotide comprising the sequence 5′-GGAACAGTTCGTCCATGGC-3′ (SEQ ID NO:1), wherein said oligonucleotide may contain one or more backbone modification and wherein said nucleotide bases are methylated or unmethylated.

It is to be understood that both the foregoing general description and the following detailed description are exemplary and explanatory only and are not restrictive of the claimed subject matter.

### BRIEF DESCRIPTION OF THE DRAWING

FIG. 1 shows the dissolution profiles for Examples 1 to 5.

FIG. 1a shows the initial release period for the dissolution profiles for Examples 1 to 5 in order to more clearly show the lag time in release of the active ingredient.

FIG. 2 shows the dissolution profiles for Examples 1, 4, 6 and 7.

FIG. 3 shows the dissolution profiles for Examples 2, 5, 8 and 9.

### DETAILED DESCRIPTION

### Definitions

As used herein, reference to an element by the indefinite article “a” or “an” does not exclude the possibility that more than one of the element is present, unless the context clearly requires that there is one and only one of the elements. The indefinite article “a” or “an” thus usually means “at least one.” The disclosure of numerical ranges should be understood as referring to each discrete point within the range, inclusive of endpoints, unless otherwise noted. The term “about” as used in the disclosure of numerical ranges indicates that deviation from the stated value is acceptable to the extent that the deviation is the result of measurement variability and/or yields a product of the same or similar properties.

As used herein, the term “active ingredient,” “active substance,” “active component,” “active pharmaceutical ingredient” and “active agent” have the same meaning as a component which exerts a desired physiological effect on a mammal, including but not limited to humans. Non-limiting examples of active ingredients according to the disclosure include but are not limited to drugs, supplements, dietary supplements, such as vitamins or provitamins A, B, C, D, E, PP and their esters, carotenoids, anti-radical substances, hydroxyacids, antiseptics, molecules acting on pigmentation or inflammation, biological extracts, antioxidants, cells and cell organelles, antibiotics, macrolides, antifungals, itraconazole, ketoconazole, antiparasitics, antimalarials, adsorbents, hormones and derivatives thereof, nicotine, antihistamines, steroid and non-steroid anti-inflammatories, ibuprofen, naproxen, cortisone and derivatives thereof, anti-allergy agents, antihistamines, analgesics, local anesthetics, antivirals, antibodies and molecules acting on the immune system, cytostatics and anticancer agents, hypolipidemics, vasodilators, vasoconstrictors, inhibitors of angiotensin-converting enzyme and phosphodiesterase, fenofibrate and derivatives thereof, statins, nitrate derivatives and anti-anginals, beta-blockers, calcium inhibitors, anti-diuretics and diuretics, bronchodilators, opiates and derivatives thereof, barbiturates, benzodiazepines, molecules acting on the central nervous system, nucleic acids, peptides, anthracenic compounds, paraffin oil, polyethylene glycol, mineral salts, antispasmodics, gastric anti-secretory agents, clay gastric dressings and polyvinylpyrrolidone, aluminum salts, calcium carbonates, magnesium carbonates, starch, derivatives of benzimidazole, and combinations of the foregoing. Orally disintegrating tablets in certain embodiments of the instant disclosure may also comprise a glucuronidation inhibitor, for example, piperine.

Non-limiting examples of active ingredients according to the present disclosure include dextromethorphan, fexofenadine, guaifenesin, loratadine, sildenafil, vardenafil, tadafil, olanzapine, risperdone, famotidine, loperamide, zolmitriptan, ondansetron, cetirizine, desloratadine, rizatriptan, piroxicam, paracetamol, phloro-glucinol, nicergoline, metopimazine, dihydroergotamine, mirtazapine, clozapine, zolmitriptan, prednisolone, levodopa, carbidopa, lamotrigine, ibuprofen, oxycodone, diphenhydramine, ramosetron, tramadol, zolpidem, fluoxetine, hyoscyamine, and combinations thereof.

Placebo dosage forms are also within the scope of the instant disclosure. In the case of a placebo, the active substance may be a substance in the excipient of the instant formulation that satisfies the goal of a placebo treatment, which is to objectively impart no specific activity for the condition being treated.

As used herein, “w/w %” and “wt %” means by weight as a percentage of the total weight.

As used herein, the weight ratio of the low pH polymer to high pH polymer given as (wt low pH polymer:wt high pH polymer) means the relative weight of the low pH polymer to the relative weight of the high pH polymer. For example, a coating which comprises 15 wt % low pH polymer and 60 wt % high pH polymer has a weight ratio of 1:4 (wt low pH polymer:wt high pH polymer).

In one aspect, a dosage form comprises a capsule containing a fill composition, and a coating over the capsule. The coating comprises a low pH polymer and a high pH polymer, the low pH polymer dissolving in phosphate buffer solution at a pH of greater than or equal to pH 4.5, and less than pH 7, and the high pH polymer dissolving in phosphate buffer solution at a pH of greater than or equal to 6.8. The weight ratio (dry) of the low pH polymer to the high pH polymer is from 1:20 to 20:1 (wt low pH polymer:wt high pH polymer). Preferably said ratio is from 1:15 to 15:1, more preferably from 1:10 to 10:1, or from 1:6 to 6:1, or from 1:5 to 5:1, most preferred from 1:4 to 4:1 (wt low pH polymer:wt high pH polymer). In another embodiment, the weight ratio of the low pH polymer to high pH polymer in the coating on a dry basis may be from 1:3 to 3:1, and may be from 1:2 to 2:1 (wt low pH polymer:wt high pH polymer).

The low pH polymer is a polymer that dissolves in phosphate buffer solution at pH of greater than or equal to 4.5 and below 7. An appropriate phosphate buffer solution may be prepared by dissolving 6.8 g potassium dihydrogen orthophosphate and 0.9 g sodium hydroxide in 1 litre water, and adjusting the pH to 6.8±0.02 using 1M hydrochloric acid. Dissolution of the polymer is evaluated using a USP apparatus 2 with a paddle speed of 50 rpm at 37° C. using phosphate buffer at the appropriate pH. The low pH polymer only begins to dissolve or disintegrate when the dosage form has exited the stomach and entered the small intestine. More preferably, the low pH polymer dissolves at a pH of greater than or equal to 5, and even more preferably greater than 5.5. The low pH polymer is fully dissolved in phosphate buffer solution at a pH of less than pH 7, more preferably less than pH 6.8. By “dissolves at a pH of greater than X” means that the polymer does not dissolve and is solid below pH X, and dissolves or disintegrates at a pH of greater than X. By “dissolves at a pH of greater than X and less than Y” means that the polymer does not dissolve and is solid below pH X, and dissolves or disintegrates at a pH of greater than X, and is fully dissolved or disintegrated at a pH of Y or less than Y.

Polymers suitable for use as the low pH polymer include: polymethacrylates such as copolymers of methyl methacrylate and methacrylic acid, copolymers of ethyl acrylate and methacrylic acid; cellulose derivatives having a carboxylic acid group such as carboxymethylethylcellulose (CMEC), cellulose acetate trimellitiate (CAT), hydroxypropylmethyl cellulose phthalate (HPMCP), and hydroxypropylmethyl cellulose acetate succinate (HPMCAS); polyvinyl derivatives such as polyvinyl acetate phthalate (PVAP), cellulose acetate phthalate (CAP); and shellac. The pH at which the polymer dissolves may be adjusted by varying the relative amounts of acidic groups and other substituents on the polymer. Commercially available low pH polymers are shown in Table 1.

TABLE 1

| | | | pH at | |
|---|---|---|---|---|
| | | | which | |
| | | | Polymer | Manufac- |
| Trade Name | Low pH Polymer | Form | Dissolves | turer |
| L30 D-55 | Poly(methacrylic | 30% | >5.5 | Evonik |
| Eudragit ® | acid, ethylacrylate) | Aqueous | | |
| | 1:1 | dispersion | | |
| L100-55 | Poly(methacrylic | Powder | >5.5 | Evonik |
| Eudragit ® | acid, ethylacrylate) | | | |
| | 1:1 | | | |
| L100 | Poly(methacrylic | Powder | >6.0 | Evonik |
| Eudragit ® | acid, methyl | | | |
| | methacrylate) 1:1 | | | |
| L12.5 | Poly(methacrylic | 12.5% | >6.0 | Evonik |
| Eudragit ® | acid, methyl | organic | | |
| | methacrylate) 1:1 | solution | | |
| COATERIC | polyvinyl acetate | Powder | >5 | Colorcon |
| | phthalate | | | |
| Opadry ® | polyvinyl acetate | Aqueous | >5 | Colorcon |
| | phthalate | mixture | | |
| Aquatec | cellulose acetate | Aqueous | >6.2-6.5 | FMC |
| | phthalate | suspension | | |
| Eastman ™ | Cellulose acetate | Powder | >6.2-6.5 | Eastman |
| C-A-P | phthalate | | | |
| Cellulose | | | | |
| Ester | | | | |
| CAT | Cellulose acetate | powder | >5.2 | Eastman |
| | trimellitate | | | |
| HP 50, 55, | Hydroxypropyl- | powder | >5, 5.5 | Shin Estu |
| 55S | methyl cellulose | | | |
| | phthalate | | | |
| HP50F, | Hydroxypropyl- | powder | >5, 5.5 | Shin Estu |
| 55FS | methyl cellulose | | | |
| | phthalate | | | |
| HPMCP50, | Hydroxypropyl- | powder | >5, 5.5 | Shin Estu |
| HPMCP55 | methyl cellulose | | | |
| | phthalate | | | |
| Aqoat ® L, M | Hydroxypropyl- | powder | >5.5 and 6 | Shin Etsu |
| | methyl cellulose | | | |
| | acetate succinate | | | |
| Duodcell | Carboxymethylethyl | powder | >5 | Freund |
| | cellulose | | | |

Especially preferred materials for the low pH polymer are copolymers of methacrylic acid and ethyl acrylate, sold under the trade name Eudragit® L30 D-55 (Evonik-Nutrition & Care GmbH, Essen, Germany).

The high pH polymer is a polymer that dissolves in a phosphate buffer solution at pH of greater than 6.8. Dissolution of the polymer is evaluated using a USP apparatus 2 with a paddle speed of 50 rpm at 37° C. using a phosphate buffer solution at pH 6.8. The high pH polymer therefore only begins to dissolve, if at all, when the dosage form has reached the distal intestinal region. More preferably, the high pH polymer dissolves at a pH of greater than or equal to 7.0, or may dissolve at a pH of greater than 7.2.

Polymers suitable for use as the high pH polymer include: polymethacrylates such as copolymers of methyl methacrylate and methacrylic acid, copolymers of ethyl acrylate and methacrylic acid, and poly(methacrylic acid, methyl acrylate, methyl methacrylate); cellulose derivatives having a carboxylic acid group such as carboxymethylethylcellulose (CMEC), cellulose acetate trimellitiate (CAT), hydroxypropylmethyl cellulose phthalate (HPMCP), and hydroxypropylmethyl cellulose acetate succinate (HPMCAS); polyvinyl derivatives such as polyvinyl acetate phthalate (PVAP), cellulose acetate phthalate (CAP); and shellac. The pH at which the polymer dissolves may be adjusted by varying the relative amounts of acidic groups and other substituents on the polymer. Commercially available polymers suitable for the high pH polymer are shown in Table 2.

TABLE 2

| | | | pH at | |
|---|---|---|---|---|
| | | | which | |
| Trade | | | polymer | Manufac- |
| Name | high pH Polymer | Form | dissolves | turer |
| S100 | Poly(methacrylic | Powder | >7 | Evonik |
| Eudragit ® | acid, methyl | | | |
| | methacrylate) 1:2 | | | |
| S12.5 | Poly(methacrylic | 12.5% | 7 | Evonik |
| Eudragit ® | acid, methyl | Organic | | |
| | methacrylate) 1:2 | solution | | |
| FS 30D | Poly(methacrylic | 30% | >7 | Evonik |
| Eudragit ® | acid, methyl | Aqueous | | |
| | acrylate, methyl | dispersion | | |
| | methacrylate) 1:10 | | | |
| | acid:ester | | | |
| Aqoat ® H | Hydroxypropylmethyl | powder | pH 6.5-6.8 | Shin Etsu |
| | cellulose acetate | | | |
| | succinate | | | |

Especially preferred materials for the high pH polymer are polymethacrylates such as poly(methacrylic acid, methyl acrylate, methyl methacrylate) sold under the trade name Eudragit® FS 30 D (Evonik Nutrition & Care GmbH, Essen, Germany).

The ratio of low pH polymer to high pH polymer in the coating is chosen to obtain the desired release profile. In operation, the low pH polymer is solid in the stomach and dissolves or disintegrates in the small intestine and/or lower gastrointestinal tract, creating pores or openings in the coating to allow ingress of GI fluid that can dissolve the capsule and allow release of the active ingredient. The high pH polymer resists dissolution or disintegration in the stomach and small intestine and dissolves more slowly (if at all) in the lower GI tract, providing structural integrity to the coating. This has the advantage of containing the fill composition, particularly when the fill composition is a sustained release fill composition. The high pH polymer thus protects against release of the active ingredient from occurring too rapidly, such as in the stomach or small intestine. In general, the weight ratio of the low pH polymer to high pH polymer in the coating on a dry basis is from 1:20 to 20:1 (wt low pH polymer:wt high pH polymer), more preferably 1:10 to 10:1, more preferably from 1:5 to 5:1, and more preferably from 1:4 to 4:1. In another embodiment, the weight ratio of the low pH polymer to high pH polymer in the coating on a dry basis may be from 1:3 to 3:1 (wt low pH polymer:wt high pH polymer), and may be from 1:2 to 2:1.

In one embodiment, the dosage form provides a release profile having a lag time of at least 30 minutes during which the amount of active ingredient released is less than 20 wt %, more preferably less than 10 wt % in phosphate buffer solution. Dissolution testing to determine the release profile is determined under the following conditions. The dissolution testing is carried out at 37° C. in a two buffer stage process (0.1M HCl initially for two hours, using a visual check for capsule rupture, before transferring the capsules to a pH 6.8 phosphate buffer stage until completion). USP apparatus 2 is used with a paddle speed of 50 rpm, media volume of 900 ml and a sample volume of 1 ml. The lag time may be achieved by a combination of varying the ratio of the low and high pH polymers and the composition of the fill formulation.

In addition to the low pH polymer and high pH polymer, the coating may contain other components.

In one aspect, the coating contains a plasticizer. Suitable plasticizers include triethyl citrate, tributyl citrate, dibutyl sebacate, triacetin, fractionated coconut oil, vegetable oil, acetylated monoglycerides, mono/di-glycerides, diethyl phthalate, dibutyl phthalate etc. A preferred plasticizer is triethyl citrate. The plasticizer may be present in the coating on a dry basis of 10 to 30 wt %.

In one aspect, the coating contains a glidant or antisticking (anticaking) agent. Suitable glidants include colloidal silicon dioxide, talc, magnesium stearate, stearic acid, and sodium lauryl sulphate. A preferred glidant is talc. The glidant may be present in the coating on a dry basis of 0 to 40 wt %.

Other components may also be present in the coating composition such as dyes, colourant or pH modifiers.

In one aspect, the coating comprises the low pH polymer in an amount of from 15 wt % to 50 wt %, the high pH polymer in an amount of from 15 wt % to 50 wt %, a plasticizer in an amount of from 5 wt % to 15 wt %, and a glidant in an amount of from 20 wt % to 40 wt % (wt % on a dry coating basis). In one aspect, the coating comprises a polymethacrylate polymer as the low pH polymer in an amount of from 15 wt % to 50 wt %, a polymethacrylate polymer as the high pH polymer in an amount of from 15 wt % to 50 wt %, triethyl citrate as a plasticizer in an amount of from 5 wt % to 15 wt %, and talc as a glidant in an amount of from 20 wt % to 40 wt % (wt % on a dry coating basis).

Capsules suitable for use in the dosage form are any capsules suitable for oral administration that dissolve or disintegrate across a range of pH as encountered in the GI tracts of humans and other animals. Exemplary capsules include gelatin capsules, hydroxypropylmethyl cellulose capsules, pullulan capsules, and starch capsules.

The fill composition contains an active ingredient and one or more excipients. Excipients suitable for use in the fill composition include all of the excipients for formulating liquid fill hard capsules including the family of polyethylene glycols (PEGs) and PEG derivatives, the mono-, di and triglycerides, sorbitan fatty acid esters such as sorbitan monooleate and sorbitan oleate (e.g., SPANs), the polysorbates, and the fatty acid esters of propylene glycol and sorbitol. The active ingredient is present in an amount suitable for the required dose.

In one aspect, the fill composition comprises a sustained release fill composition. The sustained release fill composition dissolves, disintegrates or erodes over time in response to ingress of water into the dosage form to slowly release the active ingredient over time. Excipients suitable for use in the sustained release fill composition include: polymers that swell or gel in the presence of water, such hydroxypropyl methyl cellulose, methyl cellulose, hydroxyl propyl cellulose, pectin, alginates, pregelatinised starch and other modified starches, and silicone gel; fats, waxes or other water insoluble lipids, such as triglycerides of saturated long/medium chain fatty acids; and other suitable materials for bulking and modifying drug release rates such as disintegrants.

In one embodiment, the sustained release fill composition comprises swelling or gelling polymers such as hydroxypropyl methyl cellulose (e.g., Methocel® K100) that are suspended in a water soluble or dispersible non-aqueous matrix such as polyoxylstearate (e.g., Gelucire® 48/16), or polyoxyethylene (20) sorbitan monooleate (e.g., polysorbate 80). This sustained release fill composition is designed such that on the creation of micro-pores in the coating due to solubilisation of the low pH polymer, the capsule dissolves and water is ingressed into the fill formulation and hydrates the swelling and gelling polymers which in turn modulates drug release. The release rate will be controlled by the physicochemical properties of the active ingredient (e.g. solubility, % drug load), the type and amount of the gelling/swelling polymer, the suspending matrix and the ratio of low/high pH polymers in the coat

In another embodiment, the sustained release fill composition comprises a waxy matrix characterized by the waxy matrix material being insoluble in water and having a melting temperature above 40° C. and at the same time an amount of highly water soluble material. Exemplary waxy materials include hard fats (such as Gelucire® 43/01) and long chain glycerides such as glyceryl behenate and glyercyl distearate (such as Precirol® ATO5). Exemplary highly water soluble materials include poloxamers and water-soluble methylcellulose and hydroxypropyl methylcellulose polymers (such as Methocel®. The weight ratio of the waxy material to highly water soluble material may range from 1:2 to 20:1, or may range from 1:1 to 10:1 (wt waxy material:wt highly soluble material). The sustained release formulation comprising the waxy matrix and highly water soluble material is designed such that on the creation of micro-pores in the coating due to solubilisation of the low pH polymer, the capsule dissolves and water is ingressed into the fill formulation and dissolving away the highly water soluble material creating a channel like structure to release the active ingredient within. The release rate will be controlled by the physicochemical properties of the API (e.g. solubility, % drug load), the type and amount of the high melting insoluble waxy material, the amount and solubility of the highly soluble material and the ratio of low/high pH polymers in the coat.

Dosage forms may be prepared as follows. First, capsules are filled with the fill composition, including the active ingredient. This may be performed using any suitable process.

In one aspect, the capsules are sealed or banded prior to application of the coating. Sealing or banding of the capsule allows the smoothing of the gap between the body and cap of the capsule, as such, there is no abrupt step change within the body of the capsule that can lead to catastrophic mechanical damage of the coating. Sealing or banding may be performed in any conventional manner, such as by the use of a Capsugel LEMS sealing equipment or a Qualiseal or IMA banding equipment.

Optionally, a subcoating of a water soluble coating is applied to the capsule in addition to or instead of the band. Exemplary subcoating materials include hydroxypropyl methyl cellulose, hydroxyl propyl cellulose, methyl cellulose, and starch. The coating comprising the low pH polymer and high pH polymer is than applied to the subcoating.

A coating solution or suspension may be prepared as follows. The low pH polymer and high pH polymer are mixed with a suitable solvent or liquid to form a solution or suspension. Suitable solvents include water, acetone, ethanol, and water/solvent blends.

In one aspect, the coating suspension comprises a polymethacrylate (such as Eudragit® L30 D-55 (approximately 30% dry solid)) as the low pH polymer in an amount of from 10 wt % to 35 wt %, a polymethacrylate (such as Eudragit® FS 30D (approximately 30% dry solid)) as the high pH polymer in an amount of from 10 wt % to 35 wt %, a plasticizer (such as triethyl citrate) in an amount of from 1 wt % to 3 wt %, and a glidant (such as talc) in an amount of from 2 wt % to 10 wt %; and water in an amount of from 30 wt % to 70 wt %.

The coating may be applied using any conventional process, such as by fluid bed coating and pan coating. Between 2 and 12 mg/cm² of coating, preferably 4-9 mg/cm² (polymer based) may be applied to the capsule.

It should be understood that the embodiments described herein are not limited thereto. Other embodiments of the present disclosure will be apparent to those skilled in the art from consideration of the specification and practice of the disclosed embodiments. The following examples should be considered as exemplary only, with a true scope and spirit of the present disclosure being indicated by the following claims.

### EXAMPLES

Fill Compositions

Five fill formulations were prepared in stainless steel vessels (Table 3), with caffeine used as a model active ingredient. The required quantity of lipid based excipient and HPMC was dispensed into a suitable vessel. The required quantity of caffeine was then added to the mix and mixed by spatula to wet the powder. Formulations 1-3 represent matrix formulations comprising a gelling/swelling polymer suspended in a water soluble or dispersible non-aqueous dispersion. Formulations 4-5 represent matrix formulations comprising a water soluble polymer suspended in a water insoluble high melting waxy matrix. The fill formulation was then high shear mixed for periods not exceeding 5 minutes and at least 10° C. above the melting point of any solid or semi-solid lipidic excipient contained within the formulation, until a visibly homogeneous suspension was achieved.

TABLE 3
Fill Formulation compositions

| Fill | | | | | | | Caffeine | |
|---|---|---|---|---|---|---|---|---|
| formulation | Main Carrier | | | Viscosity Modifier | | | concentration | |
| number | Excipient | % | mg | Excipient | % | mg | % | mg |
| 01 | Polysorbate | 90.2 | 360.7 | Methocel ® K100M | 7.3 | 29.3 | 2.5 | 10 |
| | 80 | | | | | | | |
| 02 | Gelucire ® | 90.2 | 360.8 | Methocel ® K100M | 7.3 | 29.2 | 2.5 | 10 |
| | 48/16 | | | | | | | |
| 03 | Kolliphor ® | 90.2 | 360.8 | Methocel ® K100M | 7.3 | 29.2 | 2.5 | 10 |
| | EL | | | | | | | |
| 04 | Gelucire ® | 82.9 | 331.5 | Methocel ® E3 | 14.6 | 58.4 | 2.5 | 10 |
| | 43/01 | | | | | | | |
| 05 | Precirol ® | 82.9 | 331.5 | Methocel ® E3 | 14.6 | 58.4 | 2.5 | 10 |
| | ATO5 | | | | | | | |

Following preparation, the fill formulations were then hand filled into size 1 gelatin Coni-Snap® capsules, to a target fill weight of 400 mg with upper limit 420 mg and a lower limit 380 mg (±7.5% target weight).

The filled capsules were then banded with a 25% gelatin banding solution using a Quali-Seal bench scale banding machine. The banded capsules were then left to air dry for a minimum of 6 hours at room temperature. Following this, the capsules were subjected to vacuum testing (<−20 mmHg) and then visually inspected for any signs of leaking or defects. Finally, the capsules were stored in double polythene bags at 2-8° C. until required for coating.

Coating Solutions

Three coating solutions based on Eudragit® L30 D-55 and Eudragit® FS 30D were prepared with the following ratio of low pH polymer (Eudragit® L30 D-55-“L polymer”) to high pH polymer (Eudragit® FS 30D-“S polymer”): 50:50 L:S polymer ratio, 25:75 L:S polymer ratio and 75:25 L:S polymer ratio (Table 4). The required quantities of Eudragit L30 and Eudragit FS30 were dispensed into a suitable vessel and stirred for a minimum of 10 minutes, using a magnetic stirring plate. Following mixing, the required quantity of sterile water was dispensed into a separate vessel. To this, the required quantity of talc was dispensed and mixed by spatula to wet the talc. The required quantity of triethyl citrate was then dispensed into the vessel containing the water and talc, and the mixture was high shear mixed for a minimum of 10 minutes, until a visually homogeneous suspension was formed. The water/talc/triethyl citrate mix was added slowly to the Eudragit L 30 and FS 30 mixture and the suspension was stirred for a minimum of 10 minutes, before being filtered through a stainless steel sieve of 500 μm. The filtered mixture was stirred until required for coating.

TABLE 4
Coating solution composition

| | Coating suspension | | | | | | | | |
|---|---|---|---|---|---|---|---|---|---|
| | composition (%) | | | Dry coat composition (%) | | | Dry coat composition (mg) | | |
| | A | B | C | A | B | C | A | B | C |
| Materials | (75/25) | (50/50) | (25/75) | (75/25) | (50/50) | (25/75) | (75/25) | (50/50) | (25/75) |
| Eudragit ® L30 D-55 | 31.25 | 20.835 | 10.42 | 46.87 | 31.25 | 15.63 | 23.435 | 15.625 | 7.815 |
| Eudragit ® FS30 D | 10.42 | 20.835 | 31.25 | 15.63 | 31.25 | 46.87 | 7.815 | 15.625 | 23.435 |
| Triethyl citrate | 2.00 | 2.00 | 2.00 | 10.00 | 10.00 | 10.00 | 5.000 | 5.000 | 5.000 |
| Talc | 5.50 | 5.50 | 5.50 | 27.5 | 27.5 | 27.5 | 13.750 | 13.750 | 13.750 |
| Sterile water | 50.83 | 50.83 | 50.83 | 0.00 | 0.00 | 0.00 | 0.000 | 0.000 | 0.000 |
| Total | 100.00 | 100.00 | 100.00 | 100.00 | 100.00 | 100.00 | 50.000 | 50.000 | 50.000 |

### Examples 1 to 9

Examples 1 to 9 were prepared by coating the filled capsules with a coating solution using a fluid bed coating machine (the Strea-1), at a coating application rate of approximately 1 mg per capsule, per minute. The capsules were weight checked periodically throughout the coating process and adjustments to the coating application rate were made if required. Coating continued until the capsules had been coated to a target of 50 mg±5 mg per capsule. The capsules were then allowed to cure for a minimum of 8 hours at room temperature, before being visually sorted to remove any defective capsules.

Table 6 shows the resulting coated capsules.

TABLE 6

| Example | Fill Formulation | Coating Suspension |
|---|---|---|
| 1 | 1 | B |
| 2 | 2 | B |
| 3 | 3 | B |
| 4 | 4 | B |
| 5 | 5 | B |
| 6 | 1 | C |
| 7 | 2 | C |
| 8 | 4 | A |
| 9 | 5 | A |

### Results

### Examples 1 to 5

Dissolution testing was performed to determine release of active from the dosage forms. The dissolution testing was carried out at 37° C. in a two buffer stage process (0.1M HCl initially for two hours, using a visual check for capsule rupture, before transferring the capsules to a pH 6.8 phosphate buffer stage until completion). USP apparatus 2 was used with a paddle speed of 50 rpm, media volume of 900 ml and a sample volume of 1 ml. Samples were taken during the pH 6.8 buffer phase, at 5, 10, 15, 30, 60, 90, 120 mins, then 3, 4, 8, 12, 18 hour time points. At least three capsules were tested for each capsule batch and per time point (n=3).

The samples were analysed on a BDS Hypersil, 0.45 μm column with a flow rate of 1 ml/min at 30° C., a run time of 5 minutes, and a detection wavelength of 275 nm. The mobile phase was 55:25:20 sodium acetate solution (0.82 mg/ml), acetonitrile, tetrahydrofuran.

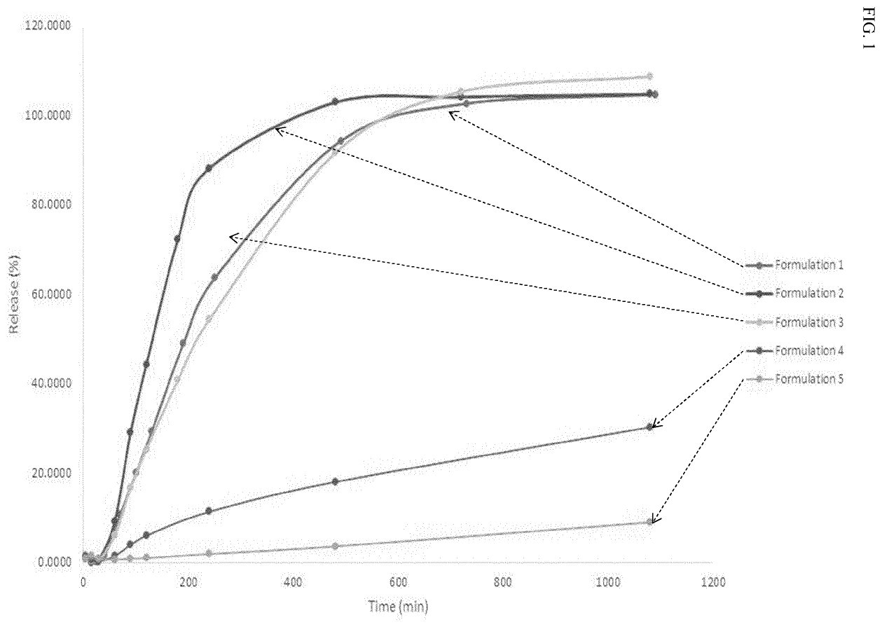

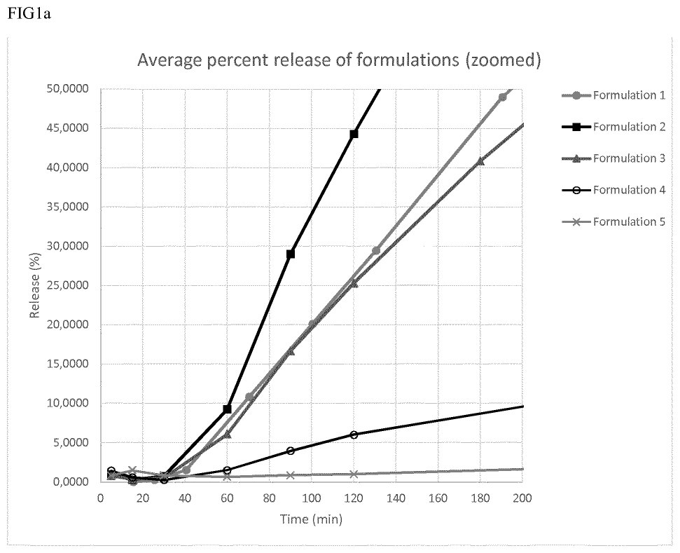

Examples 1 to 5 (the capsules coated with coating suspension B (50:50 L:S polymer ratio)) showed no visual signs of capsule rupture during 2 hour exposure to 0.1 M HCl. The capsules were then transferred to phosphate buffer at pH 6.8. The capsules showed a delay in start of caffeine release, with release initiating after approximately 1 hour (FIGS. 1 and 1a). Caffeine was then released steadily over approximately 8 hours for formulations Examples 1, 2 and 3. It is believed the release profile was due to the combined effects of the pore size generated by the L30 polymer reducing the influx and efflux of the dissolution media and HPMC gel matrix limiting the amount of formulation that was released from the capsule core. For Examples 4 and 5, the rate of release was much slower due to the change in lipid based excipient and HPMC grade.

### Examples 6 to 9

Based on the results obtained for the 50:50 coating, fill formulations 1, 2, 4 and 5 were selected for further investigation. Examples 6 and 7 were prepared using Formulations 1 and 2 coated with a 25:75 L:S Eudragit polymer ratio (suspension C) in an attempt to slow down the release profile.

Examples 8 and 9 were prepared using Formulations 4 and 5 coated with a 75:25 L:S Eudragit polymer ratio (suspension A) in an attempt to increase the release rate of active ingredient.

Examples 6 to 9 were placed in gastric media consisting of 0.1 M HCl (pH 1), and no visual signs of capsule rupture were observed after two hours of exposure. Capsules were then transferred to phosphate buffer at pH 6.8 as described above.

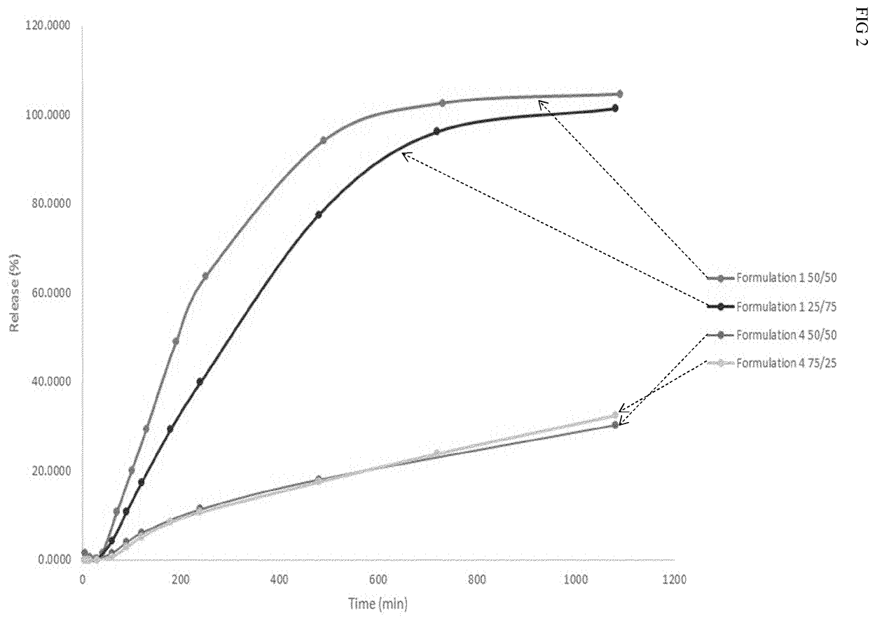

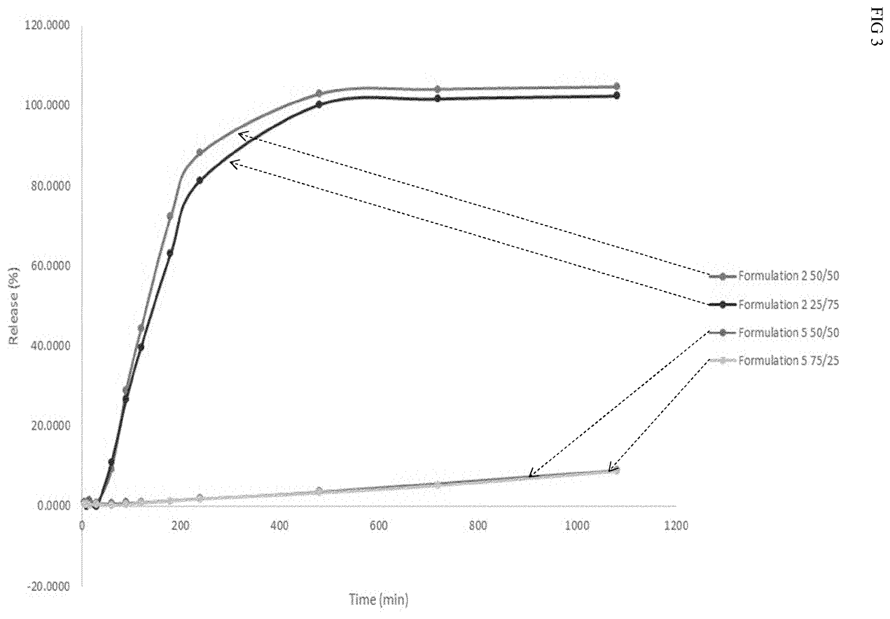

For Examples 6 and 7, increasing the relative amount of S polymer in the coating material reduced the release rate (FIGS. 2 and 3), with a greater impact shown for formulation 1 (78% release after 8 hours for 25:75 from 94% release for 50:50). For formulation 2, the decrease was less notable (100% release after 8 hours for 25:75 from 103% release for 50:50).

For Examples 8 and 9, increasing the relative amount of L polymer in the coating material had little impact on the release profile (FIGS. 2 and 3). For formulation 4, 32% release after 18 hours was shown for the 75:25 coating (Example 8) and 30% release was shown from the 50:50 coating (Example 4). For formulation 5, 9% release was shown for both coatings (Examples 5 and 9). Based on the results obtained, both the coating composition and the fill formulation composition were influential on the release rate.

As shown by FIGS. 1-3, and particularly FIG. 1a, these dosage forms are characterized by the lag time before the onset of release. This provides the advantages that when administered in vivo, the dosage form will upon reaching the distal intestine limit the release of the active ingredient in the small intestine and active ingredient release will initiate and continue in the lower gastrointestinal tract.

1. A dosage form, comprising:
a. a capsule containing a fill composition;
b. a coating over said capsule, said coating comprising a low pH polymer and a high pH polymer, said low pH polymer dissolves in a phosphate buffer solution at a pH of greater than 4.5 and is dissolved at a pH of less than pH 7, and said high pH polymer dissolves in a phosphate buffer solution at a pH of greater than 6.8, wherein a weight ratio of said low pH polymer to said high pH polymer is from 1:20 to 20:1 (wt low pH polymer:wt high pH polymer), with the proviso that said dosage form does not comprise an oligonucleotide comprising the sequence 5′-GGAACAGTTCGTCCATGGC-3′ (SEQ ID NO:1), wherein said oligonucleotide may contain one or more backbone modifications and wherein nucleotide bases of the oligonucleotide are methylated or unmethylated.

2. The dosage form of claim 1 wherein the fill composition comprises an active ingredient and said dosage form provides a lag time for release of said active ingredient of less than 20 wt % within the first 30 minutes after administration to a phosphate buffer solution at pH 6.8 and 37° C.

3. The dosage form of claim 1, wherein said low pH polymer dissolves at a pH of greater than pH 5, and dissolves at a pH of less than pH 7.

4. The dosage form of claim 1, wherein said low pH polymer is selected from polymethacrylates, polyvinyl acetate phthalate, hydroxypropylmethyl cellulose acetate succinate (HPMCAS), hydroxypropylmethyl cellulose phthalate (HPMCP), and carboxymethyl ethyl cellulose.

5. The dosage form of claim 1, wherein said low pH polymer is selected from copolymers of methyl methacrylate and methacrylic acid, and copolymers of ethyl acrylate and methacrylic acid.

6. The dosage form of claim 1, wherein said high pH polymer dissolves at a pH of greater than 7.

7. The dosage form of claim 1, wherein said high pH polymer is selected from polymethacrylates, HPMCAS and shellac.

8. The dosage form of claim 1, wherein said high pH polymer is selected from copolymers of methyl methacrylate and methacrylic acid, copolymers of ethyl acrylate and methacrylic acid, and poly(methacrylic acid, methyl acrylate, methyl methacrylate).

9. The dosage form of claim 1, wherein said weight ratio of said low pH polymer to said high pH polymer is from 1:10 to 10:1.

10. The dosage form of claim 1, wherein said coating further comprises a plasticizer.

11. The dosage form of claim 1, wherein said coating further comprises a glidant.

12. The dosage form of claim 1, wherein said coating comprises said low pH polymer in an amount of from 15 wt % to 50 wt %, said high pH polymer in an amount of from 15 wt % to 50 wt %, a plasticizer in an amount of from 5 wt % to 15 wt %, and a glidant in an amount of from 20 wt % to 40 wt % (wt % on a dry coating basis).

13. The dosage form of claim 1, wherein said capsule is banded or sealed.

14. The dosage form of claim 1, wherein said dosage form further comprises a subcoating.

15. The dosage form of claim 1, wherein said capsule comprises gelatin, HPMC, pullulan, or starch.

16. The dosage form of claim 1, wherein said fill composition is a sustained release composition.

17. The dosage form of claim 1, wherein said fill composition comprises a gelling or swelling agent suspended in a water soluble or water dispersible non-aqueous matrix.

18. The dosage form of claim 1, wherein said fill composition comprises a highly water soluble agent suspended in a water insoluble waxy matrix.

19. A method for delivering an active ingredient to the lower gastrointestinal tract of a patient comprising administering the dosage form of claim 1, wherein the fill composition of the dosage form comprises the active ingredient.


SEQUENCE LISTING (1 sequence)

SEQ ID NO: 1 - DNA, 19 bp, Artificial sequence
  targeting oligonucleotide
ggaacagttcgtccatggc